(19) United States
(12) Patent Application Publication
(10) Pub. No.: US 2024/0339209 A1
(43) Pub. Date: Oct. 10, 2024

(54) SYSTEMS AND METHODS FOR TESTING OF MEDICAL DEVICES

(71) Applicant: PRONK TECHNOLOGIES, INC., Sun Valley, CA (US)

(72) Inventors: Karl A. Ruiter, Honolulu, HI (US); Robert Edward Whitten, Sun Valley, CA (US); Julio Castro, Sun Valley, CA (US); Norman Piatek, Sun Valley, CA (US)

(21) Appl. No.: 18/629,492

(22) Filed: Apr. 8, 2024

Related U.S. Application Data
(60) Provisional application No. 63/458,074, filed on Apr. 7, 2023.

(51) Int. Cl. G16H 40/40; G16H 40/67

(52) U.S. Cl. CPC G16H 40/40; G16H 40/67

(57) ABSTRACT

Systems, devices, and methods for testing medical devices comprising: a test device configured for testing a medical device; and a user computing system in communication with the test device, where the user computing system comprises a processor having addressable memory, where the processor is configured to: display a deployed checklist for the medical device to be tested, where the deployed checklist comprises one or more test steps; display one or more persistent options for the displayed checklist, where selecting or deselecting the one or more persistent options changes the one or more test steps for the displayed checklist, and where selecting or deselecting the one or more persistent options saves the changes a next time the deployed checklist is displayed; receive input for each step of the one or more test steps of the displayed checklist; and provide instruction for the one or more test steps of the displayed checklist.

### CROSS-REFERENCE TO RELATED APPLICATIONS

This application claims priority to and the benefit of U.S. Provisional Patent Application No. 63/458,074, filed Apr. 7, 2023, the contents of which are hereby incorporated by reference herein for all purposes.

### TECHNICAL FIELD

Embodiments relate generally to medical devices, and more particularly to testing of medical devices.

### BACKGROUND

Medical devices must be regularly tested to ensure the safety of these medical devices. Manually testing a medical device may be time consuming and prone to errors. The requirements for testing of medical devices may vary based on whether a medical device will have contact with a patient or not.

### SUMMARY

A system embodiment may include: a test device configured for testing a medical device; and a user computing system in communication with the test device, where the user computing system comprises a processor having addressable memory, where the processor is configured to: display a checklist for the medical device to be tested, where the checklist comprises one or more steps; display one or more persistent options for the displayed checklist, where selecting or deselecting the one or more persistent options changes the one or more steps for the displayed checklist, and where selecting or deselecting the one or more persistent options saves the changes a next time the checklist is displayed; receive input for each step of the one or more steps of the displayed checklist; and provide information for one or more steps of the displayed checklist, where the provided information may be viewed without completing a separate step of the one or more steps of the displayed checklist.

### BRIEF DESCRIPTION OF THE DRAWINGS

The components in the figures are not necessarily to scale, emphasis instead being placed upon illustrating the principals of the invention. Like reference numerals designate corresponding parts throughout the different views. Embodiments are illustrated by way of example and not limitation in the figures of the accompanying drawings, in which:

FIG. 1 depicts a block diagram of a system for testing medical devices;

FIG. 2 depicts a top-level functional diagram of a user computing system;

FIG. 3 depicts a display of the user computer device of FIG. 2 configured for obtaining, editing, and deploying checklists;

FIG. 4 depicts the display of FIG. 2 configured for editing a checklist;

FIG. 5 depicts the display of FIG. 2 configured for performing test steps with a checklist;

FIG. 6 depicts the display of FIG. 2 configured for entering data as part of performing test steps with the checklist of FIG. 5;

FIG. 7 depicts the display of FIG. 2 configured for selecting options as part of the checklist of FIG. 5;

FIG. 8 depicts the display of FIG. 2 configured for editing checklist steps;

FIG. 9 depicts the display of FIG. 2 configured for displaying checklist steps having optional instructions;

FIG. 10 depicts the display of FIG. 2 configured for displaying the instructions from the checklist of FIG. 9;

FIG. 11 depicts the display of FIG. 2 configured for displaying a checklist exchange with filtering options;

FIG. 12 shows a high-level block diagram of a system for testing medical devices;

FIG. 13 shows a flowchart of a method for testing medical devices;

FIG. 14 shows a high-level block diagram and process of a computing system for implementing an embodiment of the system and process;

FIG. 15 shows a block diagram and process of an exemplary system in which an embodiment may be implemented; and

FIG. 16 depicts a cloud computing environment for implementing an embodiment of the system and process disclosed herein.

### DETAILED DESCRIPTION

The disclosed system and method relate to the maintenance of medical devices. Manufacturers of medical devices publish procedures for testing the medical devices. Hospitals, and other medical device owners, employ managers to ensure these procedures are followed and technicians to test the medical devices in a way that follows those procedures.

One way to ensure the procedures are followed may be to convert these procedures into checklists that technicians can go through and, step-by-step, check off what they have done. The disclosed checklists may have one or more steps. Each step may be one of pass or fail. Other steps may require entering data that is then determined to be a pass or fail based on ranges, limits, or the like. Other steps may require entering comments, where the comments may be used to determine a pass or fail. In some embodiments, each step may have a corresponding instruction that provides additional information to a technician.

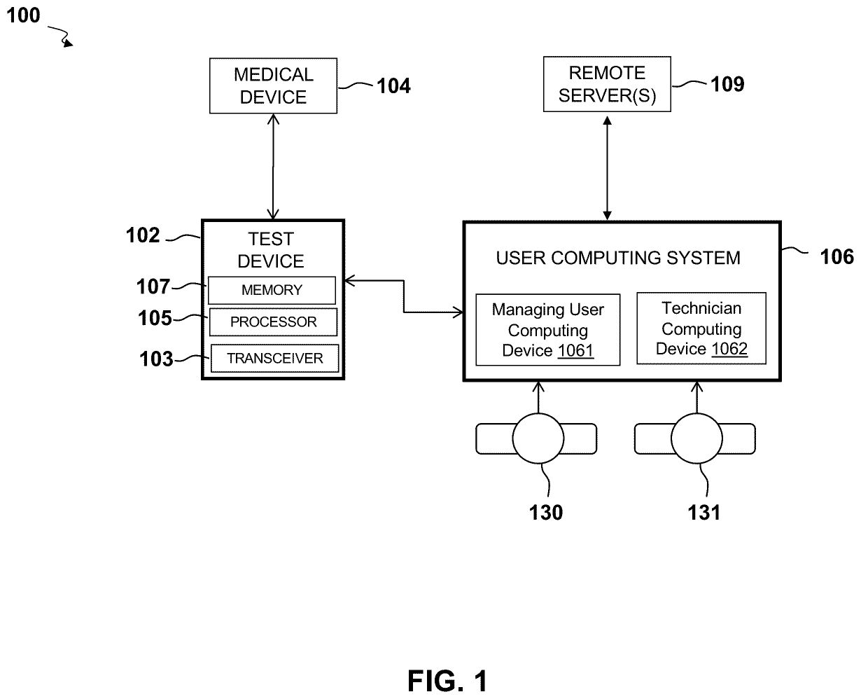

With respect to FIG. 1, a system 100 for testing medical devices 104 is shown. In one embodiment, a test device 102 may allow for generating parameter values indicating the result of one or more diagnostic tests of the performance of a medical device 104. The test device 102 may comprise a transceiver 103 and a processor 105. In some embodiments, the transceiver 103 may be a receiver and/or a transmitter. The processor 105 may have an addressable memory 107 and may execute and/or run one or more diagnostic tests for determining parameter values related to aspects of the medical device 104, such as temperature, cardiac output (CO), heart rate (HR), blood pressure (BP), SₚO₂, RR, IBP-S, IBP-D, NBP-S, NBP-D, and the like.

The test device 102 may be configured to run a plurality of tests, for example, safety test which is a subset of the over tests, on the medical device 104 to be tested. The kind, type, and criteria for each device may also differ based on the area of use. Devices in operating rooms may have different tests and corresponding pass or fail criteria than devices that are not located in patient areas. The test device 102 may be equipped to perform each test individually or to perform a number of tests sequentially. In one embodiment, the test device 102 may simulate a patient's vital signs in order to test the associated medical device 104. Each medical device 104 to be tested may have a different profile, where the profile is a set of tests to be run on a particular device and the corresponding pass or fail criteria for each test.

The disclosed system 100 and method may allow for communication between a user computing system 106, the test device 102, and/or the medical device 104. In one embodiment, the user computing system 106 operated by at least one user 130, 131 is configured for wireless communication with the test device 102. In one embodiment, the instructions in a checklist from the user computing system 106 may directly communicate with the test device (102, FIG. 1), and the user computing system 106 may obtain test results directly out of the test device (102, FIG. 1), then populate the result field directly. In other embodiments, the user computing system (106, FIG. 1) may configure the test equipment (102, FIG. 1) based on the test to be performed as part of the checklist. As an example, if there is a particular test that a technician 130 is supposed to perform, rather than having to go over to the test device and entering in a value, the disclosed method and system may enter in the value which saves time and allows the user computing system (106, FIG. 1) and/or the test device (102, FIG. 1) to operate more efficiently. The disclosed system 100 and method embodiments may also allow for communication between the user computing system 106 and a remote server 109 and/or between the test device 102 and the remote server 109 to store the test results in the remote server 109 in addition to or instead of the user computing system 106.

In some embodiments, the user computing system 106 may access the remote server 109, or a website, anywhere and allow a managing user 130 and technician users 131 to utilize the latest checklist through the remote server 109. The remote server 109 may include a CLS Remote Desktop server (CLS-RDS) where a checklist system is operated which allows the managing user 130 to obtain, edit, and deploy a checklist, and a Deployment Data Server (DDS) where allows the technician users 131 to read the deployed checklist and utilize it for testing.

In some embodiments, the user computing system 106 may include a plurality of user computing systems, including a managing user computing device 1061 and a technician computing device 1062. The managing user computing device 1061 and the technician computing device 1062 may be different hardware and different types of hardware.

The managing user computing device 1061 may be accessed by the managing user 130 who determines and deploys a checklist. The managing user computing device 1061 may be connected to the checklist system residing in the remote server 109 via internet and can remotely obtain, edit, and deploy a checklist on this checklist system. The managing user computing device 1061 may be equipped with a large screen, a keyboard, and a mouse in an office as there is significant work editing the checklist and clarity is the primary focus.

The technician user computing system 1062 may include a checklist control system and be accessed by the technician 131 who controls the test device 102 for testing the medical device 104. The deployed checklists may be downloaded from the remote server 109 onto the technician user computing system 1062 for use. The deployed checklist may be used in a patient room while standing and operating test equipment, and patient equipment. In this case, the deployed checklist can be executed on the checklist control system residing the technician user computing system 1062 even in a physical location where the technician user computing system 1062 cannot be connected to the remote server 109. The technician user computing system 1062 may have simplicity and easy operation features and thus, and in some embodiments, may not be equipped with keyboards, mice, and others.

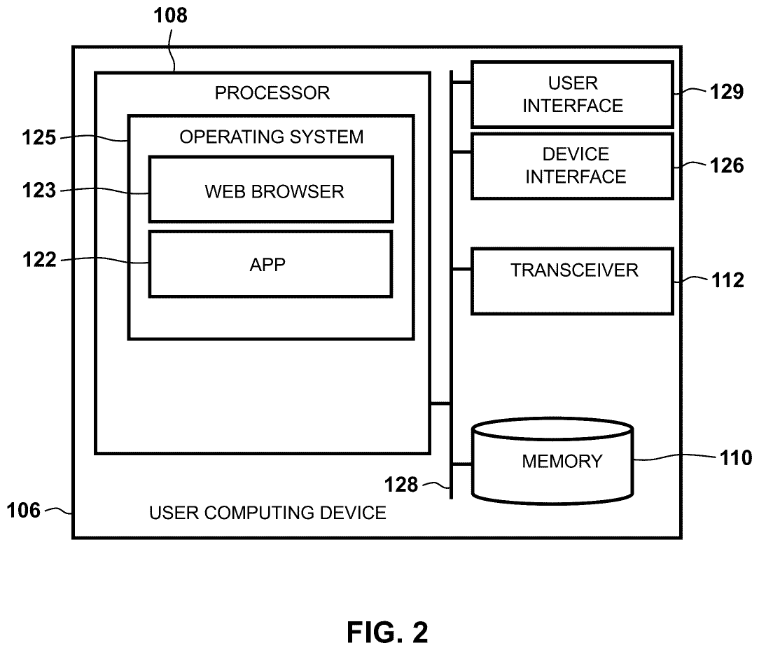

FIG. 2 depicts an example of a top-level functional block diagram of the computing device 106 of FIG. 1. The computing device 106 comprises a processor 108, such as a central processing unit (CPU), addressable memory 110, an external device interface 126, e.g., an optional universal serial bus port and related processing, and/or an Ethernet port and related processing, and an optional user interface 129, e.g., an array of status lights and one or more toggle switches, and/or a display, and/or a keyboard and/or a pointer-mouse system and/or a touch screen. Optionally, the addressable memory may be, for example: flash memory, eprom, and/or a disk drive or other hard drive. These elements may be in communication with one another via a data bus 128. In some embodiments, via an operating system 125 such as one supporting a web browser 123 and applications 122, the processor 108 may be configured to execute steps of a process establishing wireless communication with the test device 102 via a transceiver 112 of the computing device 106. In one embodiment, the transceiver 112 is a Bluetooth transceiver. In one embodiment, the user computing system 106 is a mobile device, such as a smartphone. In another embodiment, the user computing system 106 is a laptop computing device, a tablet, a smartphone, or the like. With respect to FIG. 1, the user computing system 106 may receive test results from the test device 102 and/or control aspects of the test device 102. Information regarding the test results as well as other information related to the one or more tests may be stored at the computing device 106 or at a remote server 109 in communication with the user computing system 106 and/or the test device 102.

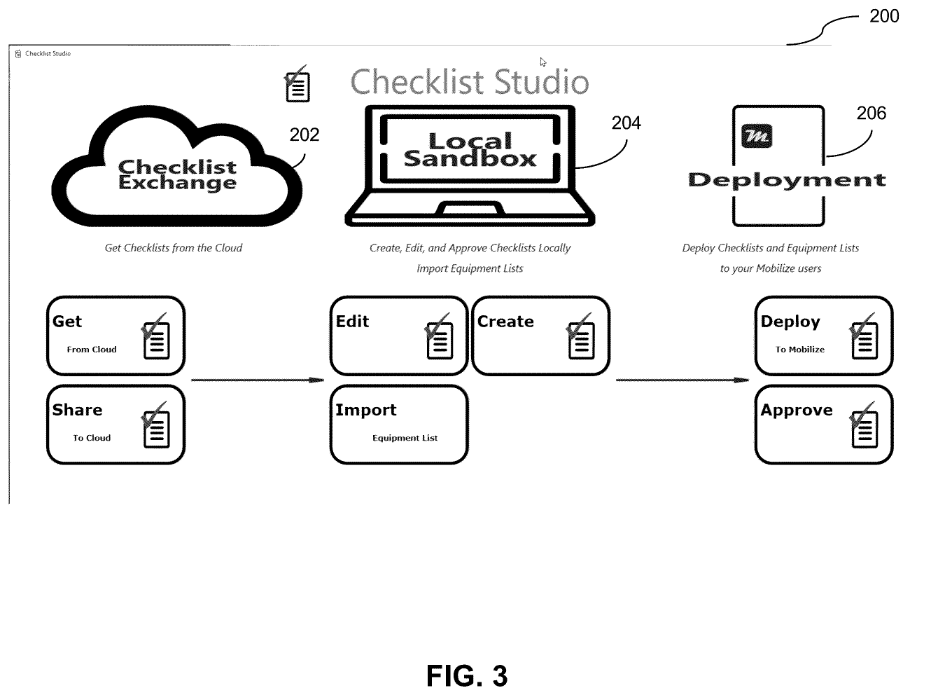

FIG. 3 depicts a display of the user computer device of FIG. 2 configured for obtaining, editing, and deploying checklists. The user interface of the user computer system (106, FIGS. 1 and 2) may include a checklist studio 200 of a checklist system. The checklist studio 200 may comprise a checklist exchange component 202, a local sandbox component 204, and a deployment component 206. The disclosed checklist studio 200 may be a tool for authoring checklists. The checklist exchange component 202 may allow the user to obtain checklists. Specifically, in the checklist exchange component 202, the user may pull down checklists that have been created from a third-party server and/or from the cloud. A wide variety of checklists may be available from one or more groups. Once selected, a checklist may be downloaded into the local sandbox 204. This checklist studio 200 may be operated in the remote server (109, FIG. 1) or may be installed and, in some embodiments, operated on the managing user computing device (1062, FIG. 1).

Specifically, in some embodiments, the pair of remote servers (109, FIG. 1) may connected to the managing user computing device (1061, FIG. 1) where the managing user may have access to and perform the steps of obtaining, editing, and deploying checklists and to the technician computing device (1062, FIG. 1) where the technician may read the deployed checklists. The first server of the remote servers may be a CLS Remote Desktop server (CLS-RDS). This allows the managing user to run the checklist studio on a virtual PC that resides on our CLS-RDS via internet schemes. The CLS-RDS may be connected to a Deployment Data Server (DDS), which is the second server of the remote servers (109, FIG. 1). The DDS may be a data server that receives the Deployment for the CLS-RDS and allows remote technician user to read it via common internet schemes, such as https, HTML, Get, etc. Thus, the user computing system may access the remote servers, or a website, anywhere and obtain the latest Deployment through the servers. In some embodiments, the service provider of the servers (109, FIG. 1) may charge the customers for access to the server system. In some embodiments, the Deployment file may be encrypted so that the service provider maintains control of the checklists. When the Deployment file is placed on the DDS, the Deployment file may be encrypted. Then, when the Deployment file is loaded into a Mobilize application, which is a tool for performing test steps, it may be decrypted.

In some embodiments, the checklists may be downloaded to a hidden location on the local machine. Afterwards, when the checklists are needed, they may not be from the cloud but instead from a local repository. Other methods of storing and obtaining checklists are possible and contemplated. In some embodiments, managing users and/or customers such as hospitals may purchase checklists. Purchases may be for individual checklists, maintenance of checklists for a set period of time, a device that is preconfigured with a number of favorite checklists, or the like. Other purchase options are possible and contemplated.

The local sandbox component 204 may allow the user to create and/or edit, checklists locally and import equipment lists. Once the checklist is created or downloaded in the local sandbox 204, a managing user may edit the checklists and otherwise make modifications to those checklists. For example, there may be additional steps that are desired but are not in the checklist or there is a desire to do certain steps differently. The managing user may use the software to edit those checklists as desired. These edits may take place in the local sandbox 204. The deployment component 206 may allow the managing user to deploy checklists and equipment lists to other users, such as technicians and/or to approve the checklist as a final checklist. Specifically, once the checklist includes all the steps that the managing user wants, the checklist may be deployed through the deployment component 206. All the checklists that an organization wants to be used by users, such as technicians, may be wrapped up into a file called a deployment file. This deployment file may be accessed by technicians on their respective user computing systems (106, FIG. 1).

The deployment component 206 may also allow the managing user to make a modification to the deployment and/or approve the checklist as a final checklist. Specifically, when a checklist is written or modified via the local sandbox components 204, the checklist may be initially deployed as a draft for testing with any test results and may be marked in a way that it is clear the draft checklist has not received final approval from the managing user. In this case, the checklist and the results may be marked with the work “DRAFT,” but it may be different marking that carries the provisional meaning. During the draft deployment, the draft checklist is tested by technicians and reviewed by the managing user for approval. Once the managing user has accepted the draft checklist, the draft checklist may be approved in the deployment component 206 via the checklist studio 200, and the any draft markings may be removed. If the checklist is later modified in the local sandbox component 204, the modified checklist must be re-deployed as a draft and then re-approved in the deployment component 206. This approval function is an important feature as it gives the managing user explicit control.

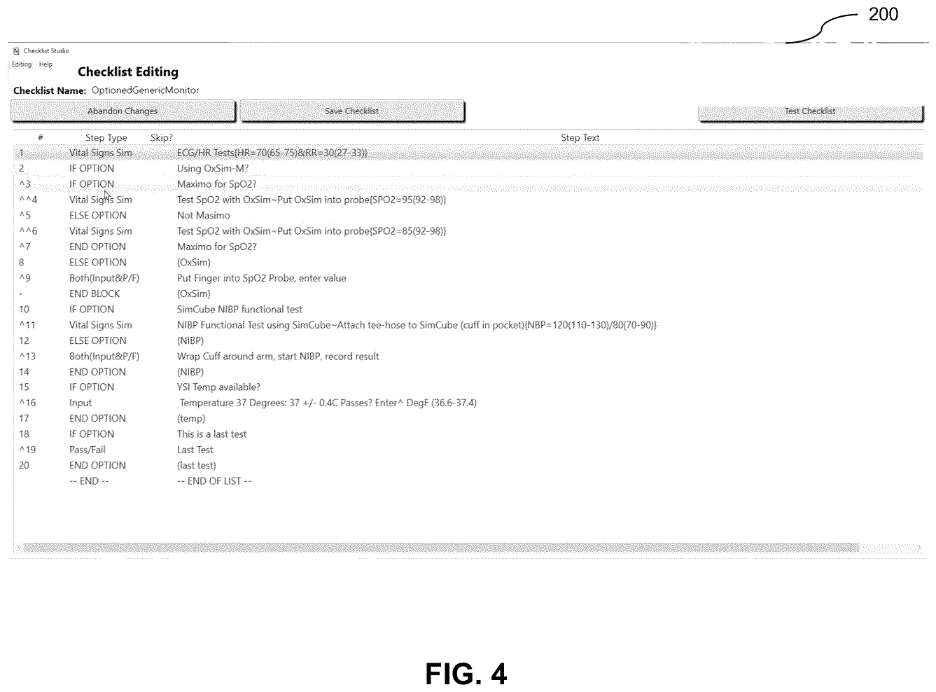

FIG. 4 depicts the display of FIG. 2 configured for editing a checklist through the local sandbox component (204, FIG. 3) of the checklist studio 200. The checklist may have one or more steps that may take place in order. In some embodiments, the order of the steps may depend on a result of a prior step, e.g., for an IF-ELSE step.

In some embodiments, the IF-ELSE block may also be presented as options to the user as, for example:
1)
IFBLOCK :: choice text
some test steps
ENDBLOCK
   This results with a Yes/No choice that the user must always make when running the checklist. No pre-selection. Yes will run the tests in the block.
2)
OPTION :: Option Name
some test steps
END OPTION
   This results in an optional group of steps that are pre-selected before the checklist is started with a checkbox or radio button named Option Name. Selection is persistent.
3)
OPTION (group name) :: Option Name 1
some test steps
END OPTION
OPTION (group name) :: Option Name 2
some test steps
END OPTION
   For options with the same group name, this results in a group of options where the check boxes are presented together and only one option in the group may be selected.

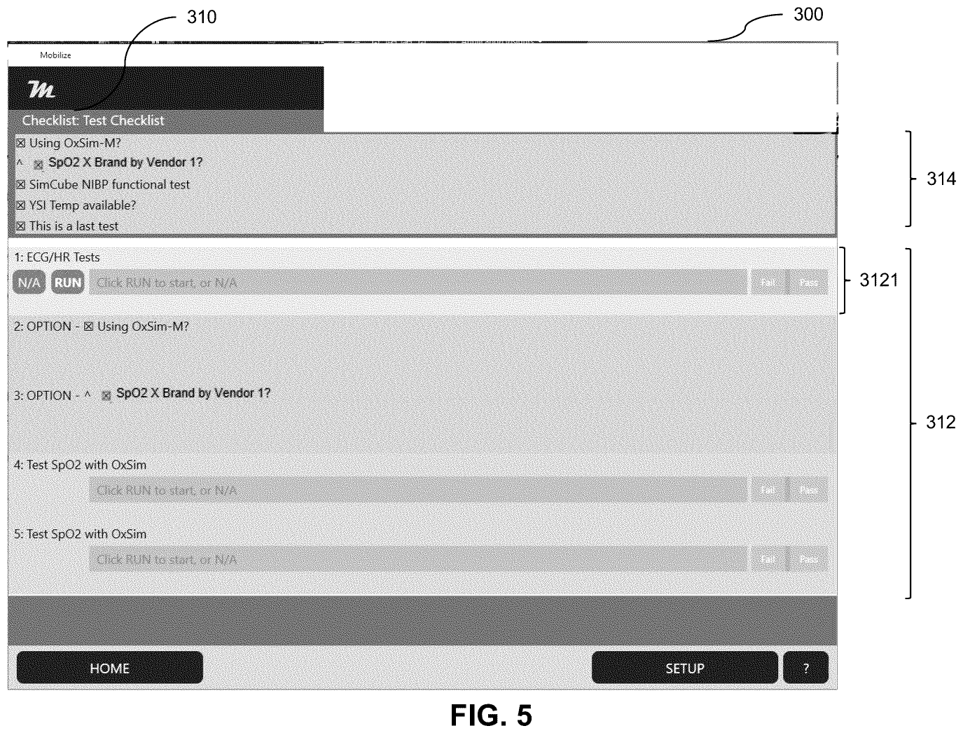

FIG. 5 depicts the display as configured for performing test steps 312 with a checklist 310. The disclosed mobilize 300 of a checklist control system may be a tool for performing the test steps 312 with the checklist 310. As described above, the deployment file including the checklist that an organization wants to be used may be accessed by technicians through the mobilize 300 on their respective user computing systems. The checklist may include persistent options 314 at the top of the checklist 310, the selection of which may persist every time the checklist 310 is opened. Individual test steps 312 may require the entry of data, the selection of an option, etc. Steps may include pass or fail values.

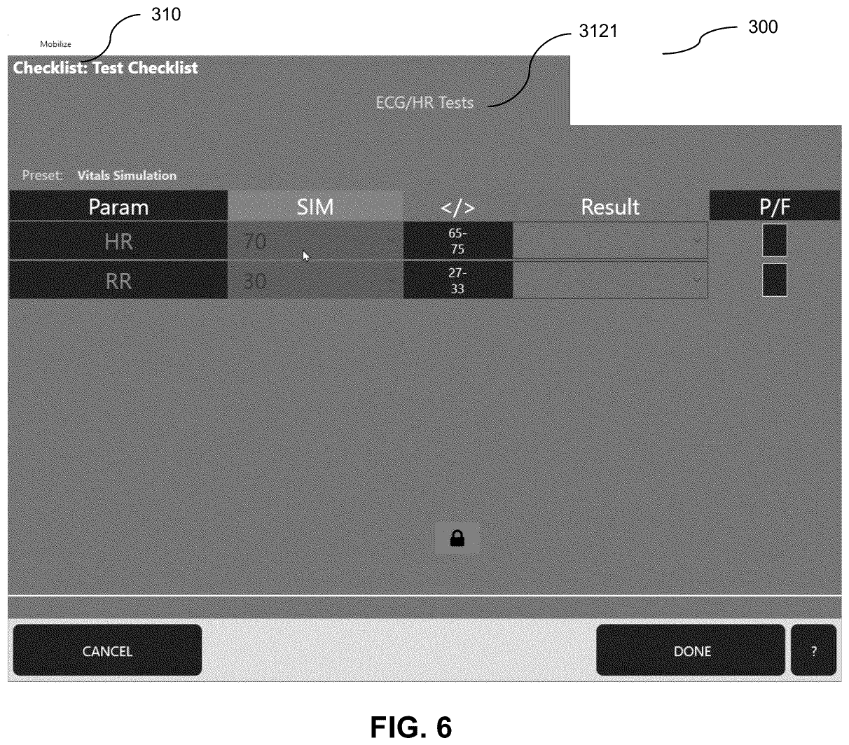

FIG. 6 depicts the display of as configured for entering data as part of performing test steps with the checklist 310 in the mobilize 300 of FIG. 5. For example, for the ECG/HR test 3121, which is regarding electrocardiogram and heart rate, a user selected heartrate may be simulated by the test device (102, FIG. 1) and then the data measured by the medical device (104, FIG. 1) may be entered on the mobilize 300 through the user computing system (106, FIG. 1) to ensure that the medical device (104, FIG. 1) is operating correctly. For example, the test device (102, FIG. 1) may simulate a user selected heartrate of 70. If the medical device (104, FIG. 1) shows a heart rate between 65-75 then the test results may be entered as either the heart rate value or a pass value in the Result or P/F section. If the medical device (104, FIG. 1) shows a heart rate lower than 65 or higher than 75 then the test may be entered as either the heart rate value or a failure value. Multiple heart rate values may be simulated and entered for each test step 3121.

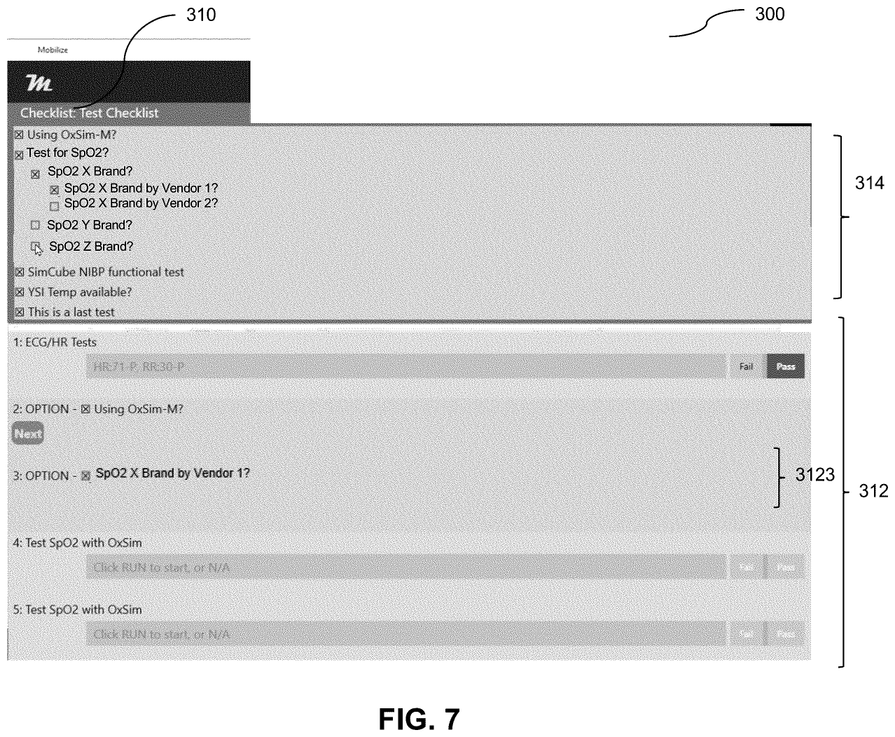

FIG. 7 depicts the display as configured for selecting options as part of the checklist of FIG. 5. In FIG. 7, when the test for SpO2 is selected, the specific devices for the SpO2 test may be presented in the indented option layers and may be selected or deselected. For example, the option for the SPO₂ pulse oximeter from X brand may be selected and the SPO₂ pulse oximeter from Y brand and Z brand may be deselected in the persistent options 314 if they are not the listed brand. In this case, there may be additional indented layers under the selected device for selecting proceeding types of the selected device. For example, since the testing of the selected device, the SPO₂ pulse oximeter from X brand, may proceed differently according to the vendor of the selected device, Vendor 1 or Vendor 2, the options for selecting Vendor 1 or Vendor 2 may be provided to proceeding the selected device in a corresponding way. The corresponding step 3123 of checklist steps 312 may then be automatically updated based on this selection, such as to follow steps for the testing of a device from the X brand for SPO₂ for a patient monitor.

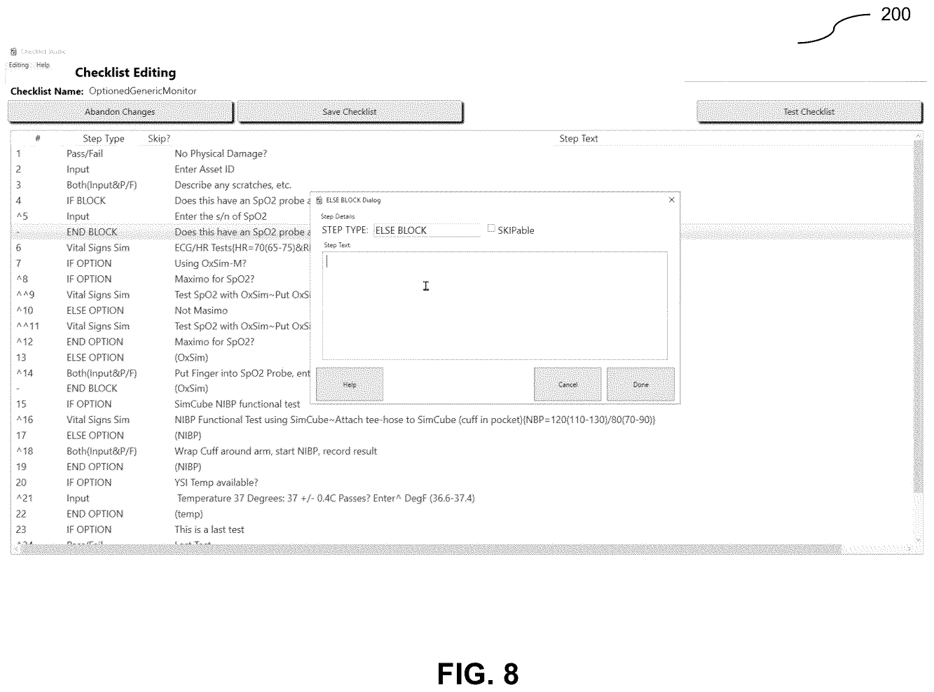

FIG. 8 depicts the display as configured for editing checklist steps according to another embodiment. The steps in a checklist may be edited by a user, such as a managing user, based on desired procedures. For example, an ELSE block may be added to show the steps to occur if a prior IF block does not return a true value. Other step types may include: pass/fail; input; input and pass/fail; END block; Vital signs simulator; IF option; and ELSE block. Other step types are possible and contemplated.

In some embodiments, a conditional statement may be added to the checklist. For example, an SPO₂ pulse oximeter may be placed on a tip of a finger to measure oxygen saturation in blood. Meanwhile, medical devices, such as this SPO₂ pulse oximeter, may be various since the medical devices in different hospitals may be purchased from different vendors. Specifically, a patient monitor may be purchased with different SPO₂ pulse oximeters from different vendors. Many hospitals standardize on a vendor. For example, Hospital I may have Brand X patient monitors that have all Brand A SPO₂ pulse oximeters while Hospital II may have all the same Brand X patient monitors, but they all have Brand B SPO₂ pulse oximeters. Each one of those vendors, Brand A and Brand B, has a series of test steps. However, the test steps may vary by brand.

The disclosed system and method may include a conditional statement that may include an IF block. In a situation where Brand X patient monitors are being tested via the disclosed checklist, there may be a portion where the device tests SPO₂. The disclosed system and method may ask if the SPO₂ pulse oximeter is made by Brand A or Brand B. Depending on the answer, the system is configured to determine a checklist based on a series of test steps to be executed.

In some embodiments, an ELSE statement may be provided for an alternate series of test steps to be executed. For example, if a user does not have a SPO₂ pulse oximeter from Brand A, then the user must have a SPO₂ pulse oximeter from Brand B and provide the steps for testing a SPO₂ pulse oximeter from Brand B.

These conditions may be based on, not only on how the medical device (104, FIG. 2) is configured, but also on what test devices (102, FIG. 1) are available. That is, if a user (131, FIG. 1) is using a certain brand of test device (102, FIG. 1), then the user (131, FIG. 1) does the test a certain way, and if the user (131, FIG. 1) does not have the same brand of test device (102, FIG. 1), then the user does the test a different way. In the disclosed system and method, both of those cases are supported.

In some embodiments, the disclosed IF blocks may be based on an equipment configuration that is to be tested, what test equipment are available, and other such conditions. For places, such as hospitals, with medical equipment to be tested, there may be the same brands of equipment available throughout the hospital and so every time a technician executes a checklist, the technician may want to make the same decisions regarding testing and testing blocks. Once the technician makes the decision, the technician may not want to make it again, and they may want their prior choices to be persistent. A technician may have a certain configuration of test equipment. Once the technician selects that test equipment, the technician would like it to stay persistent. The difference between IF/ELSE block configurations in FIG. 8 and option configurations (314, FIG. 7) in FIG. 7 is that the option configurations are persistent. For example, if a hospital has Philips monitors and they all, or mostly all, have the same type of SpO2 devices from the same vendor, the persistent options may be used since the technician does not need to make the same decision regarding testing every time executing a checklist. However, if they have a mixture of SpO2 devices from different vendors, Vendor 1 and Vendor 2, in their Philips monitors, then the IF/ELSE block may be utilized since the technician needs to change a decision regarding testing according to the currently used device.

In the disclosed system and method, one or more persistent options may be presented at the top of each checklist. In some embodiments, the one or more persistent options may be radio buttons that allow the technician to configure the checklist ahead of time and persistently. These persistent options allow the technician to pre-configure the checklist for the medical device (104, FIG. 1) that is to be tested and/or the test device (102, FIG. 1) used to test that medical device (104, FIG. 1). In some embodiments, the persistent options for pre-configuration may always be visible to a technician. The technician may change the selections in the one or more persistent options anytime a change is desired by the technician and this change will persist.

The disclosed system and method with the disclosed one or more persistent options makes the test device (102, FIG. 2) and/or the user computing system (106, FIG. 1) more efficient as it does not require making repeated selections. As an example, a hospital may have five-hundred or more patient monitors that may all have the same brand of SPO₂ pulse oximeter. Using the disclosed system and method, the technician may configure the test device (102, FIG. 1), make those selections ahead of time, and those selections may then stay persistent and be visible.

In one embodiment, if another technician logs in to another user computing system (106, FIG. 1) to test another test device (102, FIG. 1) in the same hospital, the technician may have access to the same checklist as the previous technician. Each technician may be assigned a user computing system (106, FIG. 1), such as a Windows PC, iPhone, iPad, Android device, tablet, smartphone, or the like. The user computing system (106, FIG. 1) may move around with the technician as the technician goes to various medical devices (104, FIG. 1) for testing with various test devices (102, FIG. 1).

The persistent options may be available on the user computing system (106, FIG. 1) of each technician before the technician starts into a checklist. If the technician wants to make a change to the persistent options, they can do so. Alternatively, if the technician does not want to make a change to the persistent options, then the checklist will proceed with the prior steps from the last time the checklist was used.

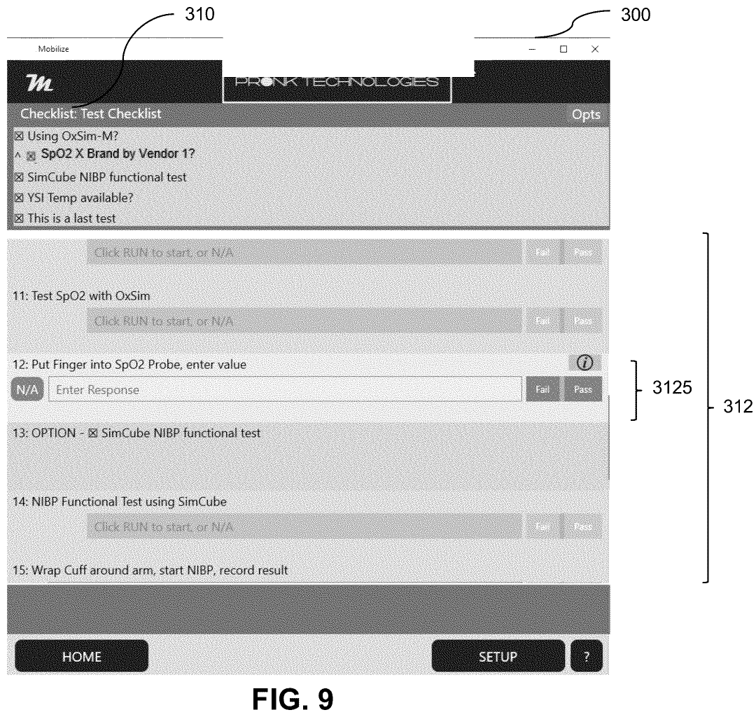

FIG. 9 depicts the display as configured for displaying checklist steps having optional instructions in the mobilize 300 of the checklist control system. If a technician using a user computing system (106, FIG. 1) presses the information icon shown as an “i” surrounded by a circle, then information may be displayed for that step.

In some embodiments, checklists 310 may include steps that include directions for a technician to follow. For example, a step 3125 may ask a technician to put their finger into an SPO₂ pulse oximeter and enter the value. However, in many circumstances the steps that a technician is supposed to perform are a lot more complicated. One approach is to code that step or test process into a large number of test steps. With this approach, the user must click through all those steps acknowledging that each step has been completed. In this case, most of these steps may just be instructions and not actually tests.

Another option, as in the disclosed method and system, is the use of a test step called an instruction. An instruction may be a large text. In some embodiments, the text may be formatted, be in a list, etc. In some embodiments, the instructions may include links to the world wide web, such as a video hosted online, a webpage, the DDS, or the like.

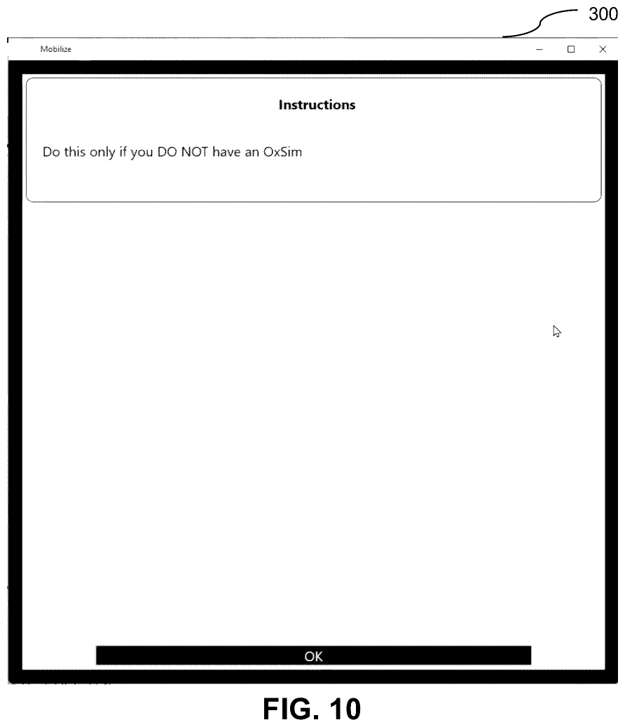

FIG. 10 depicts the display as configured for displaying the instructions from the checklist of FIG. 9 in the mobilize 300. The information is shown as plain text, but it may include formatted text, web links, audio, video, links to product manual pages, or the like. The user may exit out of the information window by pressing the “OK” button.

In some embodiments, the instructions may include one or more pages of a service manual that describes how to do a particular test. The instructions may include a link that opens the correct page of the service manual or display diagrams and/or figures in the service manual.

In some embodiments, the instruction may not automatically appear. In one embodiment, the instruction may be visible via an icon, symbol, or the like. For example, an information button may be present on steps having instructions. When a technician clicks on, or selects, the information button then the instructions may appear. As an example, many medical devices have a procedure that starts with a requirement to perform a visual inspection. The visual inspection may vary depending on the device. For example, in some devices, the technician is checking for fluids coming out; in other devices, the technician is checking for cracks; in other devices, the technician is checking hoses or supplies; and the like. If a technician has already tested several of the same devices in the same day, the technician may not need detailed steps of text to perform this initial visual inspection. However, if the technician has never performed a test on a specific medical device or medical device brand, has not done a specific test for an extended period, such as a year, etc., then the technician may require the instructions. The information needed by the technician may be placed in an optional instruction that the technician can bring up if desired. The information is in an optional instruction if the technician needs it. If the technician does not need the information, the information is not shown.

In some embodiments, each checklist on a user computing system (106, FIG. 1) may be written by and/or approved by a managing user. Individual technicians may not have access to modify the checklists once approved. In some embodiments, the checklists may have an option for the technicians to give feedback to the managing user. For example, feedback may include comments that a checklist does not work, or it is hard to do, or there are some problems with a step in the checklist.

In the disclosed systems and methods, there is no need to code instructions as test steps. As a result, the process of walking through the checklist is more efficient as compared to a checklist where each instruction is a step. This approach also makes the processor of the user computing system (106, FIG. 1) work more efficiently as it allows for more effective data mining. For example, a user may use data mining to determine an efficiency of the medical device testing and/or an efficiency of the checklists in performing that medical device testing. By removing informational steps as steps, there is less noise to filter out in determining efficiency of testing and status of medical devices in an organization, such as a hospital.

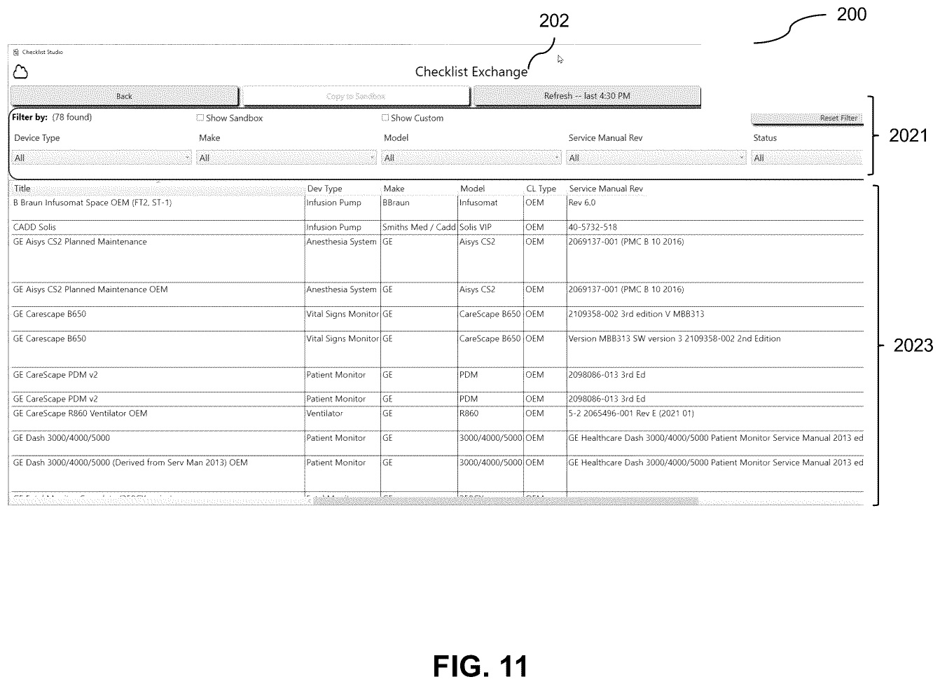

FIG. 11 depicts the display as configured for displaying the checklist exchange component 202 with filtering options 2021 in the checklist studio 200. The filtering options 2021 may be available based on metadata present in each checklist of the listed checklists 2023.

Each of the checklists 2023, in addition to the steps, has metadata. The metadata in the checklist may include the name of the manufacturer, the name of the medical device, the revision of the service manual, and the like. A managing user may sort from available checklists using one or more filters, such as manufacturers, service manual revision, and the like. This metadata information may be in a JSON format. The deployment file may be in an XML format. In another example, the deployment file may contain metadata in a JSON format.

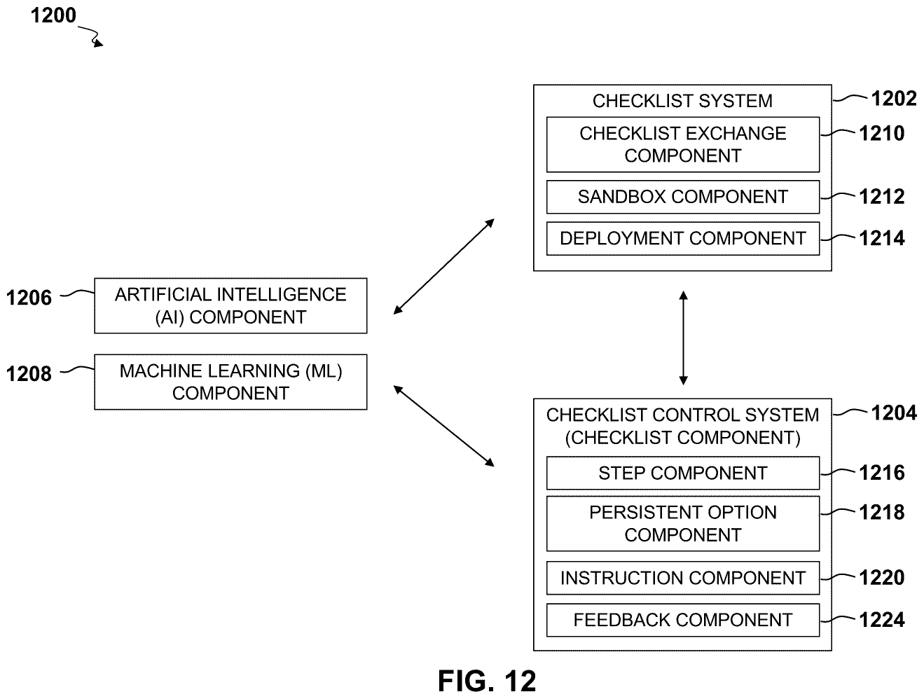

FIG. 12 shows a high-level block diagram of a system 1200 for testing medical devices. The system 1200 may include a checklist system 1202, a checklist control system 1204 including a checklist component, an artificial intelligence component 1206, and/or a machine learning component 1208.

The checklist system 1202 may include a checklist exchange component 1210, a sandbox component 1212, and a deployment component 1214. The checklist exchange component 1210 may allow a user, such as a managing user, the ability to search for, select, and/or download one or more checklists for testing one or more medical devices (104, FIG. 1). The sandbox component 1212 may allow a user, such as a managing user, the ability to view, edit, and/or save one or more checklists selected from the checklist exchange component 1210. For example, the managing user may make changes to a checklist based on the needs of the organization, such as a hospital. The deployment component 1214 may allow a user, such as a managing user, the ability to create a deployment file that contains the one or more checklists reviewed in the sandbox component 1212. The deployment file may be loaded onto user computing systems (106, FIG. 1) for each technician that may then use the one or more checklists with one or more test devices (102, FIG. 1) to test one or more medical devices (104, FIG. 1). The checklist system 1202 may be operated in a pair of remote servers (109, FIG. 1), including a CLS Remote Desktop server (CLS-RDS) and a Deployment Data Server (DDS). In some embodiments, the checklist system 1202 may be installed and operated on the managing user computer device 1061.

The checklist control system 1204, including the checklist component, may include a step component 1216, a persistent option component 1218, an instruction component 1220, and a feedback component 1224. The checklist component of the checklist control system 1204 may include one or more checklists that may be accessed via the checklist exchange component 1210 of the checklist system 1202. The step component 1216 may include one or more steps that may be included as part of a checklist. The steps in the step component 1216 may include a pass or fail entry, entry of data corresponding to pass or fail criteria, or the like.

The persistent option component 1218 may include one or more options that may be presented at a top of a checklist. The persistent option component 1218 may include, for example, radio buttons that allow certain options to be selected or deselected. These options may relate to, for example, specific steps in the step component 1216 for certain brands of medical devices (104, FIG. 1) to be tested. Different brands of medical devices may have different procedures for testing. An organization, such as a hospital, may have a uniform inventory of medical devices from a certain brand. By selecting an option in the persistent option component 1218, the steps in the step component 1216 of the checklist may be modified. The selections in the persistent option component 1218 may be saved and available to a specific technician and/or all technicians in the organization, such as a hospital, so that each future medical test uses the previously selected options. This avoids the need for a technician to manually select the brand or follow steps that are not needed as part of a checklist of the checklist component of the checklist control system 1204.

The instruction component 1220 may include one or more instructions for one or more steps of the step component 1216. The instruction component 1220 may include plain text, formatted text, audio, video, links to websites, links to user manuals, or other information relating to a step of a checklist of the checklist component 1204. The instruction component 1220 may be optionally accessed to provide additional material to a technician testing a medical device (104, FIG. 1) on an as-needed basis. By moving instructions from steps to additional material, data on the steps may be more readily mined for making improvements. These improvements may be made with the AI component 1206 and/or ML component 1208 in some embodiments. In other embodiments, a managing user may analyze data from the steps of the step component 1216 to make improvements to the checklists of the checklist component of the checklist control system 1204.

The feedback component 1224 may include options for a user, such as a technician, to provide feedback on one or more steps of the step component 1216 for a checklist of the checklist component of checklist control system 1204. This feedback may be used by a user, such as a managing user, to make changes to a checklist via the sandbox component 1212 of the checklist system 1202. Feedback may include comments that a step of the step component 1216 is not working properly, takes too long to do, is not possible, etc.

As described above, the user computing system (106, FIG. 1) may include the managing user computing device (1061, FIG. 1) and the technician computing device (1062, FIG. 1). The managing user computing device (1061, FIG. 1) may be connected to the checklist system 1202 residing in the remote server (109, FIG. 1) via internet and may remotely obtain, edit, and deploy a checklist on the checklist system 1202. The technician computing device (1062, FIG. 1) may read the deployed checklists through the remote server, but the deployed checklists may be downloaded from the remote server onto the technician computing device. The deployed checklist can be locally executed on the checklist control system which resides on the technician user computing system. This is important because the task of executing the checklist may happen when the technician computing device is located in a physical location where it cannot connect to the remote server. During the draft deployment phase of a checklist lifecycle, the managing user may also use the checklist control system 1204 to test the draft checklist as part of the approval process. In some embodiments, a single user computing system may be used to both login to the checklist system 1202 and utilize the checklist control system 1204, but the present disclosure is not limited thereto.

In some embodiments, the checklist system 1202 and the checklist control system 1204 may be included in at least portion of the user computing system (106, FIG. 1). In some embodiments, the user computing system may include a plurality of user computing systems. In this case, at least one of the user computing system may be operated by the managing user, and at least one of the user computing system may be operated by the technicians.

In some embodiments, a machine learning (ML) and/or artificial intelligence (AI) may be used to determine appropriate checklists for each technician based on the devices owned by an organization and the checklists edited and deployed by a managing user. For example, when tests may be performed in any order, the order of the test may be varied to determine an order that has a highest accuracy rate and/or takes a shortest amount of time to execute. A first test may naturally lead into a second test whereas the first test may require additional setup to move to a third test and then back to the second test. Specifically, the AI component 1206 and/or the ML component 1208 may be used to improve and/or suggest improvements to the process for testing one or more medical devices (104, FIG. 1) via one or more test devices (102, FIG. 1) controlled by a user computing system (106, FIG. 1), which is operated by a technician (131, FIG. 1). The AI component 1206 and/or the ML component 1208 may be used to perform A-B tests of checklists in order to determine a most efficient and/or most effective approach for testing medical devices (104, FIG. 1); to determine possible issues with tests for medical devices (104, FIG. 1); to recognize that a particular step and/or a particular machine is failing every time and then alert, highlight, or notate this potential issue to the managing user; to send a weekly notification to the managing user with a list of all failures and/or potential issues; and the like. Other possibilities with the AI component 1206 and/or the ML component 1208 are possible and contemplated.

In some embodiments, the ML component 1208 and/or the AI component 1206 may be used to determine possible issues with tests for medical devices (104, FIG. 1). For example, if a step takes significantly longer for one medical device as compared to similar medical devices, then that medical device may be highlighted for possible issues. In some embodiments, the disclosed system and method may recognize, such as via the ML component 1208 and/or the AI component 1206, that a particular step and/or a particular machine is failing every time and then alert, highlight, or notate this potential issue to the managing user. In some embodiments, the disclosed system and method may send, such as via the ML component 1208 and/or the AI component 1206, a weekly notification to the managing user with a list of all failures and/or potential issues.

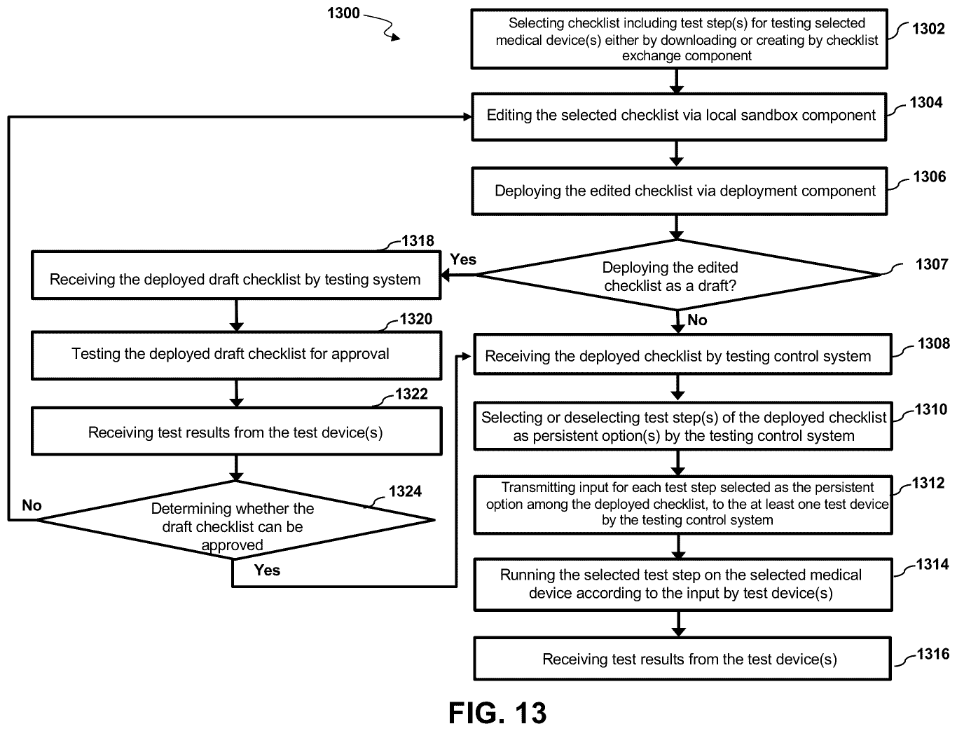

FIG. 13 shows a flowchart of a method 1300 for testing medical devices according to some embodiments. The method 1300 for testing medical devices may start with the step of obtaining, by a checklist system (1202, FIG. 12) of a user computing system, a checklist including at least one test step for testing a selected medical device (104, FIG. 1). The step of obtaining the checklist may comprises the steps of: selecting a checklist either by downloading a checklist made by a third party via a checklist exchange component (1210, FIG. 12) of the checklist system or creating a checklist by a managing user via a local sandbox component (1212, FIG. 12) of the checklist system (step 1302); editing the selected checklist via the local sandbox component (step 1304); and deploying the edited checklist via a deployment component (1214, FIG. 12) of the checklist system (step 1306). Then, once the checklist is deployed, the subsequent steps of the method 1300 may comprises: receiving, by at least one checklist control system (1204, FIG. 12) of the user computing system, the deployed checklist (step 1308); selecting or deselecting, by the persistent option component (1218, FIG. 12) of the checklist control system, at least on test step of the deployed checklist as a persistent option (step 1310); transmitting, by the checklist control system, input for each test step selected as the persistent option among the deployed checklist, to the at least one test device (102, FIG. 1); running, by at least one test device, the selected test step on the selected medical device according to the input (step 1314); and receiving, by the user computing system, test result from the test device (step 1316).

In some embodiment, the method 1300 for testing medical devices may further include the steps of drafting deployment, approval testing, and approval process. Specifically, when the managing user deploys the edited checklist via a deployment component (1214, FIG. 12) of the checklist system (step 1306), the edited checklist may be deployed as a draft, not a final checklist (step 1307). If the edited checklist is deployed as a draft, the draft checklist is tested by technician users for final approval. That is, the deployed draft checklist may be received on the checklist control system (step 1318), and the test step(s) of the deployed draft checklist may be tested using the actual test device and medical devices (step 1320). The test of the deployed draft checklist may follow the same processes as described in the steps 1310 to 1314. When the test of the deployed draft checklist is completed, and the test results may be received (step 1322), the managing user may determine whether the draft checklist can be approved (step 1324). Once approved, the draft checklist may be deployed as a final checklist and used in actual tests for medical devices. If the draft checklist is disapproved, it may be returned to the local sandbox component for additional editing.

In some embodiments, the method 1300 may be used to test a plurality of different medical devices. Even though the checklist for medical devices are different from each other, since the checklist for each medical device may be obtained from other sources, such as the cloud, which include a plurality of different checklists for the different medical device, the different medical devices can be conveniently tested by using the method 1300. In addition, the step of editing the downloaded checklist may allow the managing user to modify the checklist as needed, thereby providing the customized checklist beyond the predetermined checklist. The step of deploying the checklist may enable a plurality of technicians to utilize the checklist determined by the managing user of the organization, such as a hospital, thereby providing the consistent management of testing medical devices in the organization.

In some embodiments, the selection of at least one test step of the deployed checklist as the persistent option in the step of selecting or deselecting, may be stored, and this selection, or these changes of the test steps, may be applied again without repeating the step of selecting or deselecting the test step of the deployed checklist. This feature may provide convenience and efficiency when testing a plurality of medical devices of the same type or testing a single medical device multiple times. The step of selecting or deselecting also allows the technicians to control or customize the test steps for the specific medical device within the deployed checklist.

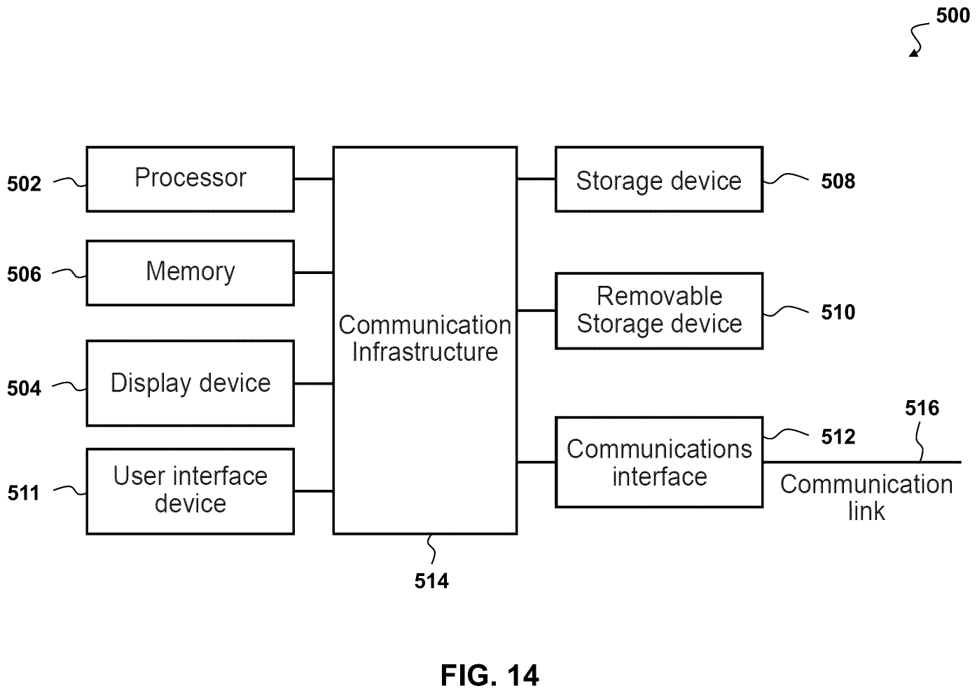

FIG. 14 is a high-level block diagram 500 showing a computing system comprising a computer system useful for implementing an embodiment of the system and process, disclosed herein. Embodiments of the system may be implemented in different computing environments. The computer system includes one or more processors 502, and can further include an electronic display device 504 (e.g., for displaying graphics, text, and other data), a main memory 506 (e.g., random access memory (RAM)), storage device 508, a removable storage device 510 (e.g., removable storage drive, a removable memory module, a magnetic tape drive, an optical disk drive, a computer readable medium having stored therein computer software and/or data), user interface device 511 (e.g., keyboard, touch screen, keypad, pointing device), and a communication interface 512 (e.g., modem, a network interface (such as an Ethernet card), a communications port, or a PCMCIA slot and card). The communication interface 512 allows software and data to be transferred between the computer system and external devices. The system further includes a communications infrastructure 514 (e.g., a communications bus, cross-over bar, or network) to which the aforementioned devices/modules are connected as shown.

Information transferred via communications interface 514 may be in the form of signals such as electronic, electromagnetic, optical, or other signals capable of being received by communications interface 514, via a communication link 516 that carries signals and may be implemented using wire or cable, fiber optics, a phone line, a cellular/mobile phone link, an radio frequency (RF) link, and/or other communication channels. Computer program instructions representing the block diagram and/or flowcharts herein may be loaded onto a computer, programmable data processing apparatus, or processing devices to cause a series of operations performed thereon to produce a computer implemented process.

Embodiments have been described with reference to flowchart illustrations and/or block diagrams of methods, apparatus (systems) and computer program products according to embodiments. Each block of such illustrations/diagrams, or combinations thereof, can be implemented by computer program instructions. The computer program instructions when provided to a processor produce a machine, such that the instructions, which execute via the processor, create means for implementing the functions/operations specified in the flowchart and/or block diagram. Each block in the flowchart/block diagrams may represent a hardware and/or software module or logic, implementing embodiments. In alternative implementations, the functions noted in the blocks may occur out of the order noted in the figures, concurrently, etc.

Computer programs (e.g., computer control logic) are stored in main memory and/or secondary memory. Computer programs may also be received via a communications interface 512. Such computer programs, when executed, enable the computer system to perform the features of the embodiments as discussed herein. In particular, the computer programs, when executed, enable the processor and/or multi-core processor to perform the features of the computer system. Such computer programs represent controllers of the computer system.

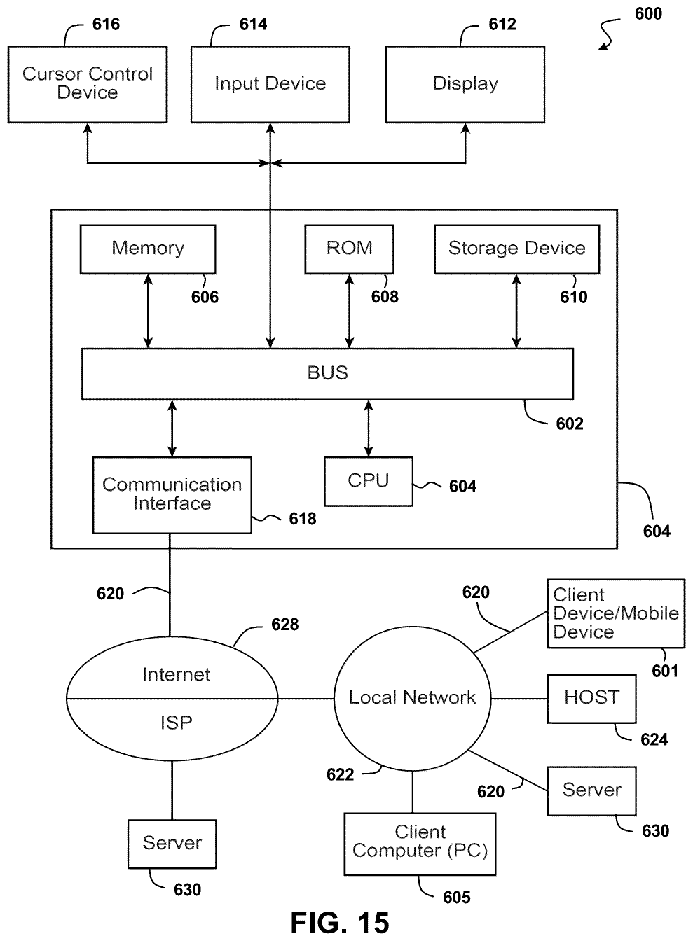

FIG. 15 shows a block diagram of an example system 600 in which an embodiment may be implemented. The system 600 includes one or more client devices 601 such as consumer electronics devices, connected to one or more server computing systems 630. A server 630 includes a bus 602 or other communication mechanism for communicating information, and a processor (CPU) 604 coupled with the bus 602 for processing information. The server 630 also includes a main memory 606, such as a random access memory (RAM) or other dynamic storage device, coupled to the bus 602 for storing information and instructions to be executed by the processor 604. The main memory 606 also may be used for storing temporary variables or other intermediate information during execution or instructions to be executed by the processor 604. The server computer system 630 further includes a read only memory (ROM) 608 or other static storage device coupled to the bus 602 for storing static information and instructions for the processor 604. A storage device 610, such as a magnetic disk or optical disk, is provided and coupled to the bus 602 for storing information and instructions. The bus 602 may contain, for example, thirty-two address lines for addressing video memory or main memory 606. The bus 602 can also include, for example, a 32-bit data bus for transferring data between and among the components, such as the CPU 604, the main memory 606, video memory and the storage 610. Alternatively, multiplex data/address lines may be used instead of separate data and address lines.

The server 630 may be coupled via the bus 602 to a display 612 for displaying information to a computer user. An input device 614, including alphanumeric and other keys, is coupled to the bus 602 for communicating information and command selections to the processor 604. Another type or user input device comprises cursor control 616, such as a mouse, a trackball, or cursor direction keys for communicating direction information and command selections to the processor 604 and for controlling cursor movement on the display 612.

According to one embodiment, the functions are performed by the processor 604 executing one or more sequences of one or more instructions contained in the main memory 606. Such instructions may be read into the main memory 606 from another computer-readable medium, such as the storage device 610. Execution of the sequences of instructions contained in the main memory 606 causes the processor 604 to perform the process steps described herein. One or more processors in a multi-processing arrangement may also be employed to execute the sequences of instructions contained in the main memory 606. In alternative embodiments, hard-wired circuitry may be used in place of or in combination with software instructions to implement the embodiments. Thus, embodiments are not limited to any specific combination of hardware circuitry and software.

The terms “computer program medium,” “computer usable medium,” “computer readable medium”, and “computer program product,” are used to generally refer to media such as main memory, secondary memory, removable storage drive, a hard disk installed in hard disk drive, and signals. These computer program products are means for providing software to the computer system. The computer readable medium allows the computer system to read data, instructions, messages or message packets, and other computer readable information from the computer readable medium. The computer readable medium, for example, may include non-volatile memory, such as a floppy disk, ROM, flash memory, disk drive memory, a CD-ROM, and other permanent storage. It is useful, for example, for transporting information, such as data and computer instructions, between computer systems. Furthermore, the computer readable medium may comprise computer readable information in a transitory state medium such as a network link and/or a network interface, including a wired network or a wireless network that allow a computer to read such computer readable information. Computer programs (also called computer control logic) are stored in main memory and/or secondary memory. Computer programs may also be received via a communications interface. Such computer programs, when executed, enable the computer system to perform the features of the embodiments as discussed herein. In particular, the computer programs, when executed, enable the processor multi-core processor to perform the features of the computer system. Accordingly, such computer programs represent controllers of the computer system.

Generally, the term “computer-readable medium” as used herein refers to any medium that participated in providing instructions to the processor 604 for execution. Such a medium may take many forms, including but not limited to, non-volatile media, volatile media, and transmission media. Non-volatile media includes, for example, optical or magnetic disks, such as the storage device 610. Volatile media includes dynamic memory, such as the main memory 606. Transmission media includes coaxial cables, copper wire and fiber optics, including the wires that comprise the bus 602. Transmission media can also take the form of acoustic or light waves, such as those generated during radio wave and infrared data communications.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, an EPROM, a FLASH-EPROM, any other memory chip or cartridge, a carrier wave as described hereinafter, or any other medium from which a computer can read.

Various forms of computer readable media may be involved in carrying one or more sequences of one or more instructions to the processor 604 for execution. For example, the instructions may initially be carried on a magnetic disk of a remote computer. The remote computer can load the instructions into its dynamic memory and send the instructions over a telephone line using a modem. A modem local to the server 630 can receive the data on the telephone line and use an infrared transmitter to convert the data to an infrared signal. An infrared detector coupled to the bus 602 can receive the data carried in the infrared signal and place the data on the bus 602. The bus 602 carries the data to the main memory 606, from which the processor 604 retrieves and executes the instructions. The instructions received from the main memory 606 may optionally be stored on the storage device 610 either before or after execution by the processor 604.

The server 630 also includes a communication interface 618 coupled to the bus 602. The communication interface 618 provides a two-way data communication coupling to a network link 620 that is connected to the world wide packet data communication network now commonly referred to as the Internet 628. The Internet 628 uses electrical, electromagnetic or optical signals that carry digital data streams. The signals through the various networks and the signals on the network link 620 and through the communication interface 618, which carry the digital data to and from the server 630, are exemplary forms or carrier waves transporting the information.

In another embodiment of the server 630, interface 618 is connected to a network 622 via a communication link 620. For example, the communication interface 618 may be an integrated services digital network (ISDN) card or a modem to provide a data communication connection to a corresponding type of telephone line, which can comprise part of the network link 620. As another example, the communication interface 618 may be a local area network (LAN) card to provide a data communication connection to a compatible LAN. Wireless links may also be implemented. In any such implementation, the communication interface 618 sends and receives electrical electromagnetic or optical signals that carry digital data streams representing various types of information.

The network link 620 typically provides data communication through one or more networks to other data devices. For example, the network link 620 may provide a connection through the local network 622 to a host computer 624 or to data equipment operated by an Internet Service Provider (ISP). The ISP in turn provides data communication services through the Internet 628. The local network 622 and the Internet 628 both use electrical, electromagnetic or optical signals that carry digital data streams. The signals through the various networks and the signals on the network link 620 and through the communication interface 618, which carry the digital data to and from the server 630, are exemplary forms or carrier waves transporting the information.

The server 630 can send/receive messages and data, including e-mail, program code, through the network, the network link 620 and the communication interface 618. Further, the communication interface 618 can comprise a USB/Tuner and the network link 620 may be an antenna or cable for connecting the server 630 to a cable provider, satellite provider or other terrestrial transmission system for receiving messages, data and program code from another source.

The example versions of the embodiments described herein may be implemented as logical operations in a distributed processing system such as the system 600 including the servers 630. The logical operations of the embodiments may be implemented as a sequence of steps executing in the server 630, and as interconnected machine modules within the system 600. The implementation is a matter of choice and can depend on performance of the system 600 implementing the embodiments. As such, the logical operations constituting said example versions of the embodiments are referred to for e.g., as operations, steps or modules.

Similar to a server 630 described above, a client device 601 can include a processor, memory, storage device, display, input device and communication interface (e.g., e-mail interface) for connecting the client device to the Internet 628, the ISP, or LAN 622, for communication with the servers 630.

The system 600 can further include computers (e.g., personal computers, computing nodes) 605 operating in the same manner as client devices 601, where a user can utilize one or more computers 605 to manage data in the server 630.

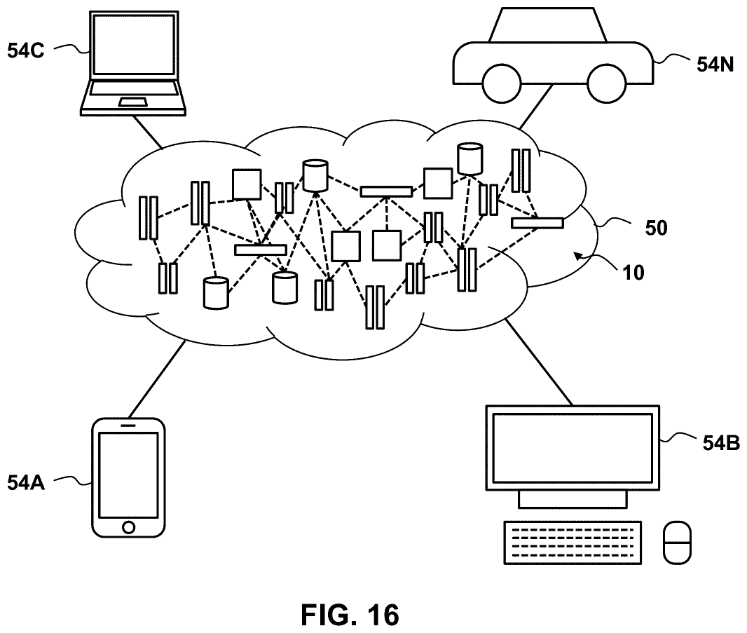

Referring now to FIG. 16, illustrative cloud computing environment 50 is depicted. As shown, cloud computing environment 50 comprises one or more cloud computing nodes 10 with which local computing devices used by cloud consumers, such as, for example, personal digital assistant (PDA), smartphone, smart watch, set-top box, video game system, tablet, mobile computing device, or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 16 are intended to be illustrative only and that computing nodes 10 and cloud computing environment 50 can communicate with any type of computerized device over any type of network and/or network addressable connection (e.g., using a web browser).

It is contemplated that various combinations and/or sub-combinations of the specific features and aspects of the above embodiments may be made and still fall within the scope of the invention. Accordingly, it should be understood that various features and aspects of the disclosed embodiments may be combined with or substituted for one another in order to form varying modes of the disclosed invention. Further, it is intended that the scope of the present invention is herein disclosed by way of examples and should not be limited by the particular disclosed embodiments described above.

What is claimed is:

1. A system for testing medical devices, the system comprising:
a test device (102) configured for testing a medical device;
a remote server (109) configured to provide a checklist system (1202) where at least one checklist is provided by a third party or created by a user, each of the at least one checklist including at least one test step for the medical device; and
a user computing system (106) configured to communicate with the test device and the remote server, the user computing system configured to:
select a checklist from the at least one checklist on the checklist system;
deploy the selected checklist as a deployed checklist on the checklist system;
determine at least one test step of the deployed checklist by selecting or deselecting at least one test step of the deployed checklist on a checklist control system;
transmit input for each of the determined test step of the deployed checklist to the test device so that the test device runs the determined test step to test the medical device according to the input; and
receive a test result from the test device.

2. The system of claim 1, wherein the checklist system includes:
a checklist exchange component where a checklist made by the third party is provided and a checklist created by a user can be shared;
a local sandbox component where the checklist made by the third party is edited or a checklist is created by a user and where a checklist from the at least one checklist is selected; and
a deployment component where the selected checklist is deployed for testing the medical device.

3. The system of claim 2, wherein the deployment component further includes an option for deploying the selected checklist as a draft.

4. The system of claim 3, wherein when the selected checklist is deployed as a draft, the user computing system performs the steps of:
determining at least one test step of the deployed draft checklist;
transmitting input for each of the determined test step of the deployed draft checklist to the test device so that the test device runs the determined test step to test the medical device according to the input; and
receiving a test result from the test device.

5. The system of claim 4, wherein the deployment component further includes an option for approving the deployed draft checklist based on the test result using the deployed draft checklist.

6. The system of claim 1, wherein the remote server allows the user computing system to access the checklist system and to operate the checklist system via an internet connection.

7. The system of claim 1, wherein the remote server allows the user computing system to install the checklist control system on the user computing system and to operate the checklist control system on the user computing system in a physical location where the user computing system cannot be connected to the remote server.

8. The system of claim 1, wherein the determination of the at least one test step of the deployed checklist includes:
displaying the at least one test step of the deployed checklist in a persistent option panel; and
determining the at least one test step of the deployed checklist as at least one persistent option by selecting or deselecting the at least one test step of the deployed checklist in the persistent option panel,
wherein the at least one test step determined as the persistent option saves the changes a next time the deployed checklist is used.

9. The system of claim 1, wherein the checklist control system displays each of the at least one test step of the deployed checklist with instructions regarding each of the at least one test step or an icon linked to the instructions,
wherein the instructions include at least one of texts, web links, audio, video, and links to product manual pages.

10. The system of claim 9, wherein the user computing system comprises a plurality of user computing systems including: a managing user computing device that operates the checklist system and a technician computing device that operates the checklist control system.

11. A system for testing medical devices comprising:
a test device configured for testing a medical device; and
a user computing system in communication with the test device, wherein the user computing system comprises a processor having addressable memory, wherein the processor is configured to:
display a deployed checklist for the medical device to be tested, wherein the deployed checklist comprises one or more test steps;
display one or more persistent options for the displayed checklist, wherein selecting or deselecting the one or more persistent options changes the one or more test steps for the displayed checklist, and wherein selecting or deselecting the one or more persistent options saves the changes a next time the deployed checklist is displayed;
receive input for each step of the one or more test steps of the displayed checklist; and
provide instruction for the one or more test steps of the displayed checklist.

12. The system of claim 11, further comprising a remote server configured to provide a checklist system where at least one checklist is provided by a third party or created by a user and a checklist control system where the deployed checklist and the one or more persistent options are displayed.

13. The system of claim 12, wherein the checklist system includes:
a checklist exchange component where a checklist made by the third party is provided and a checklist created by a user can be shared;
a local sandbox component where the checklist made by the third party is edited or a checklist is created by a user and where a checklist from the at least one checklist is selected; and
a deployment component where the selected checklist is deployed for testing the medical device.

14. The system of claim 13, wherein the deployment component further includes:
an option for deploying the selected checklist as a draft; and
an option for approving the deployed draft checklist based on a test result using the deployed draft checklist.

15. A method for testing medical devices, the method comprising:
obtaining, by a checklist system connected to a user computing system, a checklist including at least one test step for testing a selected medical device by:
selecting a checklist either by obtaining a checklist made by a third party via a checklist exchange component of the checklist system or creating a checklist by a user via a local sandbox component of the checklist system;
editing the selected checklist via the local sandbox component; and
deploying the edited checklist via a deployment component of the checklist system;
receiving, by a checklist control system residing on the user computing system, the deployed checklist;
selecting or deselecting, by the checklist control system, at least one test step of the deployed checklist as a persistent option;
transmitting, by the checklist control system, input for each test step selected as the persistent option among the deployed checklist, to the at least one test device;
running, by at least one test device, the selected test step on the selected medical device according to the input; and
receiving, by the user computing system, test result from the test device.

16. The method of claim 15, wherein the step of deploying the edited checklist include:
determining whether the edited checklist is deployed as a draft; and
deploying the edited checklist as a draft if the edited checklist is determined to be deployed as a draft, wherein after deploying the edited checklist as a draft, the method further comprises testing the deployed draft checklist.

17. The method of claim 16, wherein the step of testing the deployed draft checklist is performed by:
receiving, by the checklist control system, the deployed draft checklist;
selecting or deselecting, by the checklist control system, at least one test step of the deployed draft checklist;
transmitting, by the checklist control system, input for each test step selected among the deployed draft checklist, to the at least one test device;
running, by the at least one test device, the selected test step on the selected medical device according to the input based on the deployed draft checklist; and
receiving, by the user computing system, test result from the test device.

18. The method of claim 18, after the step of testing the deployed draft checklist, further comprising determining whether the deployed draft checklist can be approved,
wherein if the deployed draft checklist is approved, the approved checklist is deployed and used for testing medical devices, and if the deployed draft checklist is disapproved, the disapproved draft checklist is returned to the step of editing and further edited.

19. The method of claim 15, wherein the step of selecting or deselecting at least one test step of the deployed checklist includes displaying each of the at least one test step of the deployed checklist with instruction regarding each of the at least one test step or an icon linked to the instruction.

20. The method of claim 15, wherein the checklist system is provided by a remote server, and the user computing system communicate with the remote server via internet,
wherein the checklist control system resides on the user computing system and is executed in a physical location where the user computing system cannot be connected to the remote server.
